(19) United States
(12) Patent Application Publication
(10) Pub. No.: US 2007/0286830 A1
(43) Pub. Date: Dec. 13, 2007

(54) Aluminum compositions of enhanced efficacy having high HPLC Bands III and IV

(75) Inventors: Zijun Li, Westfield, NJ (US); Mark Rerek, Scotch Plains, NJ (US); Kevin Janak, Ossining, NY (US)

Correspondence Address: ARTHUR J. PLANTAMURA;GENERAL CHEMICAL PERFORMANCE PRODUCTS LLC., 90 EAST HALSEY ROAD, PARSIPPANY, NJ, 07054, US

(73) Assignee: Reheis, Inc.

(21) Appl. No.: 11/452,101

(22) Filed: Jun. 13, 2006

(51) Int. Cl. A61K 8/26

(52) U.S. Cl. 424/68

(57) ABSTRACT

Novel aluminum antiperspirant compositions of enhanced efficacy activated by both heat and acid are produced comprising
basic aluminum salts of having the formula:

Al₂(OH)₆₋ₐXₐ
wherein X is Cl⁻, Br⁻, l⁻, or NO₃⁻, a is from abut 1.1 to about 1.7, with basic aluminum chloride being the preferred salt.

When analyzed by SEC-HPLC at about 10% concentration (i.e., by 2% Al) in water, these compositions exhibit an SEC-HPLC Band I area of less than 10%, a Band III area of at least 20%, with a Band III/II area ratio of at least 0.7, a Band IV area of at least 20%, and have at least 60% aluminum species contained in Bands III and Band IV.

This invention relates to novel aluminum antiperspirant compositions of enhanced efficacy that are activated by both heat and acid and have a high HPLC Band III as well as a high HPLC Band IV and to the processes for the preparation of the compositions. The processes of the present invention include the addition of an acid such as AlCl₃ to the aluminum solutions having high HPLC Band III prepared by both direct and indirect processes, as well as the preparation of aluminum sesquichlorohydrate solutions directly through the reaction of Al powder with aqueous aluminum chloride solutions. The aluminum salt solutions derived from the processes of the invention are dried to powders and are conveniently adaptable for use in consumer-acceptable antiperspirant-formulated products including aerosols, sticks, creams and soft solids.

### BACKGROUND OF THE INVENTION

Enhanced efficacy aluminum antiperspirant salts are known and generally related to the aluminum species that are activated by either heat, as indicated by having high SEC (size exclusion chromatography)-HPLC (high performance liquid chromatography) Band III content or by acid as having high Band IV content at lower Al/Cl ratio. These compositions are typically different from conventional antiperspirant salts that have low quantities of Band III, with Band III/II area ratio of less than 0.1 or 0.2, as well as low Band IV of less than 20%. In the evaluation of “Bands” and “peaks”, as applied in the characterization for antiperspirant properties, typically in the Band reference, Bands I, II, III and IV of one system correspond to peaks 1, 2, 3, 4 and 5 of the peak system, more specifically, Band I corresponds to peak 1 plus peak 2, Band II corresponds to peak 3, Band III corresponds to peak 4 and Band IV corresponds to peak 5. Enhanced efficacy aluminum antiperspirant salts have an HPLC Band III/II area ratio of 0.5 or higher with at least 70% and preferably at least 80% of aluminum contained in Bands II and III. Enhanced antiperspirant salts typically have a Band III content of at least 20%, and usually at least 30%, of the total aluminum contained in all the peaks as measured by peak area. In contrast, conventional non-enhanced antiperspirant salts have Band III content in the range of about 10% and Band III/II area ratio from 0.1 to 0.2 or less. Typically, the enhanced aluminum salts activated by heat, that are characterized by the presence of high HPLC Band III or high Band III/II ratio, are prepared by the dilution and heat treatment of 50% ACH (aluminum chlorohydrate) solution and quickly drying the solutions to powders because of the rapid reverting back of Band III to Band II when such are in aqueous solutions, particularly at concentrations of greater than 20%.

Prior art patents describing the enhanced aluminum salts include British Patent GB 2,048,229; U.S. Pat. No. 4,359,456; U.S. Pat. No. 4,775,528; U.S. Pat. No. 4,818,512; U.S. Pat. No. 4,859,446; U.S. Pat. No. 4,871,525; U.S. Pat. No. 4,900,534; U.S. Pat. No. 4,944,933; U.S. Pat. No. 5,356,609; U.S. Pat. No. 5,358,694; U.S. Pat. No. 5,595,729 and U.S. Pat. No. 5,626,827.

Aluminum antiperspirant salts of enhanced efficacy can also be prepared through acid activation at lower Al/Cl ratio, such as ratios of less than 1.5 as described, for example, in U.S. Pat. Nos. 5,718,876 and 6,024,945, where the basic aluminum chloride solutions with 28% to 42% solid concentration have at least 50% HPLC Band II and less than 20% Band III. Further, U.S. Pat. No. 6,902,724 describes a basic aluminum chloride (BAC) composition having a Band I relative area value of less than 5%, a Band II of 20% to 60%, a Band III of 10% to 35% and a Band IV of 15% to 50% at Al/Cl ratio of 1.2 to 1.5 comprising 30% to 42% anhydrous solid. The BAC solution is prepared by reacting aluminum powder with aluminum chloride aqueous solution at a temperature greater than 85° C. In the examples disclosed, e.g., Examples 1, 6, 7 and 8, the freshly prepared BAC solutions always have Band III of less than 20% and, in any event, no BAC solution in that patent reference appears as having a Band III area greater than 25%. Moreover, the HPLC figure in that patent focuses on a BAC solution having high Bands II and IV.

Heretofore the provision of aluminum antiperspirant compositions of enhanced efficacy that are activated by both heat and acid with high HPLC Band III and high HPLC Band IV is unknown. Accordingly, the production of aluminum antiperspirant compositions of enhanced efficacy that are activated by both heat and acid and that have both a high HPLC Band III and a high Band IV is highly desirable. The aluminum-sesquichlorohydrate solutions prepared by the direct process of the invention have higher concentration and are stable in aqueous solution for several days before being spray dried compared to solutions solely prepared by dilution and heat activation that must be spray dried immediately. The aluminum powders prepared according to the invention can be used in various finished formulations, such as aerosols, sticks, soft solids and the like.

### SUMMARY OF THE INVENTION

According to the present invention, it has been discovered novel aluminum compositions of enhanced antiperspirant efficacy that are activated by both heat and acid and are characterized by having high HPLC Band III and high HPLC Band IV, are prepared by processes (referred to for convenience as Processes “A” and “B”), by the addition of an acid such as AlCl₃ to the aluminum salt solutions having high HPLC Band III prepared either directly or indirectly. In addition, these novel aluminum salt solutions may be prepared by an alternative process referred to as Process “C,” involving the direct reaction of Al powder with the aqueous AlCl₃ solutions at elevated temperatures at Al/Cl ratios of less than 1.8. The aluminum salt solutions prepared according to the invention, from each of the Processes of A, B and C are dried to powders and are characterized by having: an HPLC Band I of less than 10%, preferably less than 5%; a Band III of at least 20%, preferably at least 30%; a Band III/II ratio of at least about 0.7, preferably at least about 0.9; and a Band IV of at least 20%, preferably at least 25% and have an Al/Cl ratio of below 1.8. The powders containing activated aluminum salts derived in accordance with the invention may be utilized in formulating aerosol, soft solid, cream and stick products, as appropriate.

Accordingly, it is an object of the present invention to provide novel basic aluminum halide antiperspirant salt powder compositions of enhanced efficacy activated by both heat and acid.

It is another object of the present invention to provide antiperspirant compositions having greater antiperspirancy and skin friendliness.

It is yet another object of this invention to provide antiperspirant compositions that contain, aluminum-only actives of highest efficacy, and that can be used in formulations including, but not limited to, aerosols and that have much lower corrosion potential than conventional acid activated actives.

### BRIEF DESCRIPTION OF THE DRAWINGS

FIG. 1 is the ²⁷Al NMR of an aluminum salt composition with an Al/Cl ratio of 1.47 prepared according to Process A of the invention.

FIG. 2 is the SEC-HPLC of an aluminum salt composition with an Al/Cl ratio of 1.49 prepared according to process B of the invention.

FIG. 3 is the SEC-HPLC of an aluminum salt composition with an Al/Cl ratio of 1.71 prepared according to process C of the invention.

### DETAILED DESCRIPTION OF THE INVENTION

The present invention is directed to novel aluminum antiperspirant compositions of enhanced efficacy activated by both heat and acid which, when analyzed by SEC-HPLC at about 10% concentration (i.e., by 2% Al) in water, exhibit an HPLC Band I area of less than 10%, preferably less than 5%; a Band III area of at least 20%, preferably 30% or more; a Band III/II ratio of at least 0.7, preferably 0.9 or more; a Band IV area of at least 20%, preferably 25% or more; and have at least 60% and preferably 70% or more aluminum species contained in Bands III and Band IV.

The basic aluminum salts of these compositions have the formula:

Al₂(OH)₆₋ₐXₐ

wherein X is Cl⁻, Br⁻, I⁻ or NO₃⁻, a is from about 1.1 to about 1.7, with basic aluminum chloride the most preferred. The acronym BAC, as used herein, and as indicated by the above formula, includes the iodide and bromide halogen ions in addition to chloride, and the nitrate ion NO₃⁻. The preferred ratio of aluminum to chloride or other ions of the BAC solution is from 1.4 to 1.6.

The invention is described in detail by reference to alternative Processes A, B and C. According to Process A, about 50% by weight BAC solution is first prepared by the standard method such as described in U.S. Pat. No. 5,718,876 which is incorporated herein by reference. The ratio of Al/Cl of the BAC solution should be above 1.5, with the preferred ratio being 1.7 to 1.9. The 50% solution is diluted, so that it comprises about 10% to about 25% by weight solution, and heated until the desired HPLC Band III/II ratio is obtained. Preferably, the solution is refluxed for 1 to 2 hours, which is then cooled, followed by the addition of an acid, such as AlCl₃, Al(NO₃)₃, HCl and HNO₃, with AlCl₃ and Al(NO₃)₃ the most preferred acids. The resultant Al salt solution is quickly dried to afford an aluminum composition having high HPLC Bands III and IV. During the spray drying of the solution, the loss of some chloride may be expected.

In an alternative process, Process B involving a direct reaction with aluminum powder, about 25%-35% by weight BAC solution is prepared first such that, initially the solution has an Al/halide ratio of about 1.75 to about 1.95 during the direct reaction between Al powder and aqueous aluminum halide solution is conducted at temperatures above 65° C. An acid, such as AlCl₃ is added to provide greater activation, and the solution is then processed through a drying operation.

In the application of a direct process, referred to herein as Process C, a 25%-40% by weight BAC solution is prepared through the reaction of aluminum powder with aqueous AlCl₃ solution at a temperature of greater than 65° C. The solution generally has a ratio of Al/Cl of about 1.2 to about 1.8, preferably about 1.4 to 1.7 with a 30% to 35% by weight concentration. This solution is found to have a room temperature stability with respect to SEC-HPLC distribution of about one week and is readily dried to produce a powder product.

A polyhydric alcohol, such as propylene glycol, may be included in the aluminum solution in any of the alternative processes. Inclusion of the polyhydric alcohol tends to impart improved stability and aid dissolution of the powder product in alcohols.

The double activated aluminum solutions can be dried to powders by any appropriate means, including spray drying, freeze drying and vacuum drying. Spray drying is the most preferred method. Furthermore, the solutions can be dried into different densities and different shapes, such as low and high density spherical particles to better suit the variety of formulations, such as soft solids and low residue sticks, to which the powders of the invention may be applied.

The aluminum antiperspirant salts of the present invention are characterized as follows:

### SEC-HPLC

The degree of the polymerization of aluminum complexes is determined by Size Exclusion Chromatography (SEC) operated via a High Performance Liquid Chromatograph (HPLC) instrument. In this technique, the highest molecular weight Al species are eluted first and are designated as Band I, which is equivalent to peaks 1 and 2; Band II (equivalent to peak 3) and Band III (equivalent to peak 4) are intermediate molecular weight Al compounds. Band IV (equivalent to peak 5) is derived from the lowest molecular weight Al complexes, including monomers and dimers. Band V is derived from the chloride. The relative area of one or more peaks is determined in order to characterize the distribution of polymeric species in the aluminum complexes that are formed. All the aluminum species are eluted in Bands I, II, III and IV and the percentage of each peak area is calculated accordingly. The aluminum antiperspirant salts according to the present invention have SEC-HPLC Band III to Band II area ratio of at least 0.7, and most preferably at least 0.9. Band IV content should be at least about 20%. At least 60%, and preferably at least about 70%, of the total SEC-HPLC band content should be contained within Band III and Band IV. These enhanced salts have a Band III content of at least 20%, and preferably at least 30%, of the total aluminum contained in all the peaks as measured by peak area.

A C₁ column (150×4.6 mm) from YMC. Inc., a Column Engineering C₁ column (100×4.6 mm) and a Lichrosorb Silica column (250×4.6 mm) connected in series is used to obtain the SEC-HPLC Chromatograms. Each sample is dissolved in deionized water to form a 2% by weight Al solution. Each sample is filtered through a 0.45μ filter and chromatographed within 15 minutes using a 0.01N nitric acid solution as the mobile phase at a flow rate of 0.75 ml/minute.

### Nuclear Magnetic Resonance Spectroscopy

²⁷Al Nuclear Magnetic Resonance (NMR) is utilized to identify the structures of different aluminum species in the double activated aluminum antiperspirant salts of enhanced efficacy. The antiperspirant salt in solution form is measured as is and the powder is dissolved in deuteriated water to form a 10% by weight solution just before the measurement. Data were collected using a Varian Inova 400 instrument at 104.2 MHz.

The invention will be further illustrated by the following Examples. In the Examples, parts are by weight unless otherwise specified. The “% A.S.” is the abbreviation of anhydrous solid excluding the bonded water in antiperspirant powder.

### EXAMPLE 1

### Preparation of Activated Aluminum Solutions Containing High HPLC Band III Through Dilution and Heat Treatment

20% by weight BAC solutions were prepared by diluting about 50% BAC solutions with water and refluxed for one hour. The results are as followed,

| | | | | | |
|---|---|---|---|---|---|
| Al/Cl ratio | 1.92 | 1.88 | 1.77 | 1.46 | 1.29 |
| Band III/II | 0.84 | 0.84 | 1.07 | 0.50 | 0.27 |
| % Band IV | 2.9 | 4.2 | 5.8 | 16.5 | 25.6 |

The data indicate no aluminum salt composition that has both high HPLC Bands III (or III/II ratio greater than 0.5) and IV (at least 20%) is obtained through dilution and heat treatment of the BAC solutions at Al/Cl ratios of 1.29 to 1.92.

### EXAMPLE 2

### Preparation of Double Activated Aluminum Salt Compositions by Process A

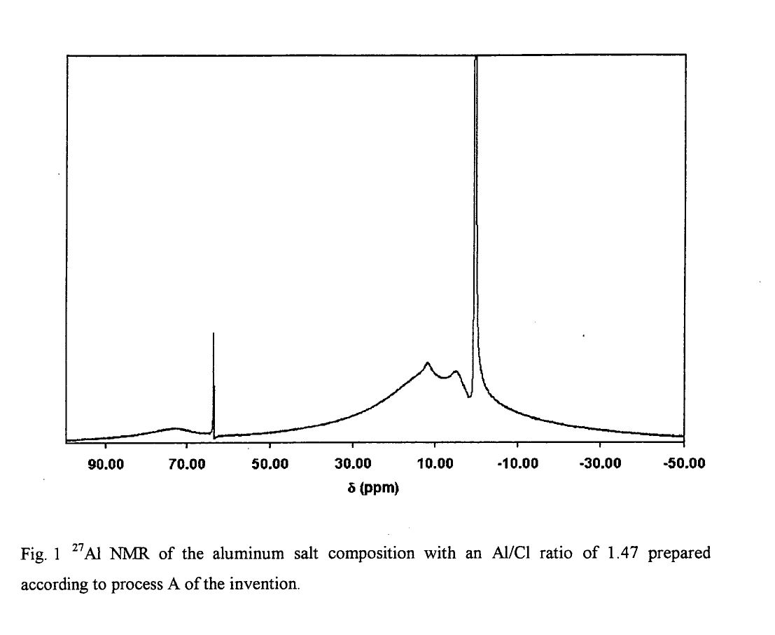

20% BAC solutions were prepared by diluting a 50% BAC solution which was refluxed for two hours. After the refluxed solutions were cooled, AlCl₃ solutions (32° Be) were added to make the double activated Al solutions with both high HPLC Bands III and IV at different Al/Cl ratios, and the solutions were spray dried. The results are shown in Table I. FIG. 1 illustrates the ²⁷Al NMR of the novel Al salt composition prepared under Experiment Number 3.

TABLE I

| Experiment | Al/Cl | | | | | |
|---|---|---|---|---|---|---|
| Number | Ratio | % A.S. | % Band I | III/II | % IV | % III + IV |
| 1 (control) | 1.89 | 83.6 | 0 | 0.90 | 8.5 | 51.8 |
| 2 | 1.57 | 80.5 | 0 | 0.92 | 27.4 | 62.1 |
| 3 | 1.47 | 79.6 | 0 | 0.91 | 32.2 | 64.4 |
| 4 | 1.34 | 77.5 | 0 | 0.82 | 31.3 | 62.3 |

### EXAMPLE 3

### Preparation of Double Activated Aluminum Salt Compositions Utilizing the Process B

(i) Double Activated Al Salt Compositions Made from 30% BAC Solution

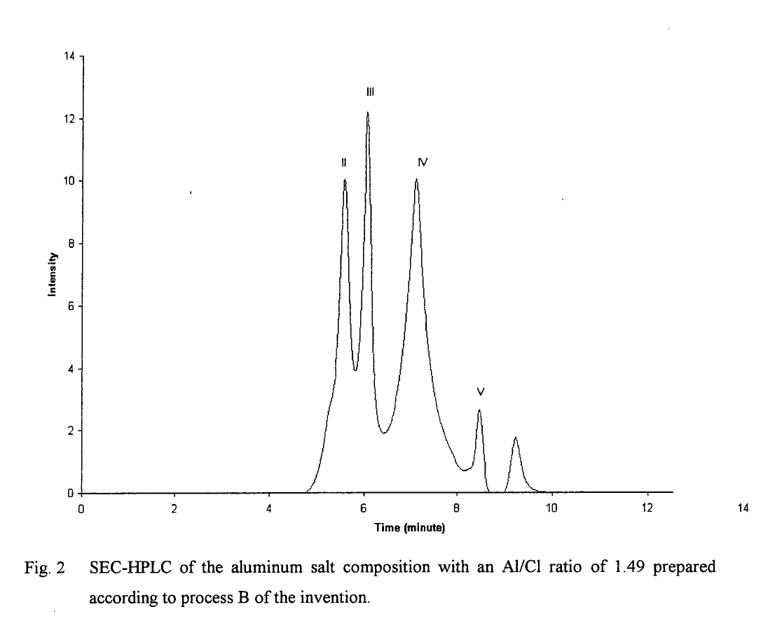

970 parts of AlCl₃ (32° Be) was mixed with 2785 parts of water and heated to about 80° C. 245 parts of aluminum powder was added and reacted at that temperature. The reaction mixture was filtered to give a clear solution, which contains 7.21% Al, 5.2% Cl with Al/Cl ratio of 1.82. Different amounts of AlCl₃ (32° Be) solution were added to the above 30% BAC solution, and the resultant solutions were spray dried the next day. Almost no loss in chloride was observed for the powders during the spray drying process. The results of two runs, Experiments Number 5 and Number 6, prepared by this process, are given in Table II. The SEC-HPLC of the Al salt composition according to Experiment Number 6 is shown in FIG. 2.

TABLE II

| Experiment | Al/Cl | | | | | |
|---|---|---|---|---|---|---|
| Number | Ratio | % A.S. | % Band I | III/II | % IV | % III + IV |
| 5 | 1.62 | 78.2 | 7.5 | 1.24 | 37.0 | 67.8 |
| 6 | 1.49 | 78.5 | 4.5 | 1.16 | 47.8 | 73.4 |

(ii) Double Activated Aluminum Salt Compositions Made from 33% BAC Solutions

580 parts of AlCl₃ (32° Be) was mixed with 1277 parts of water and heated to about 85° C. 143 parts of aluminum powder was added and reacted in about three hours. The clear solution obtained after filtration has 8.27% Al and 6.03% Cl with Al/Cl ratio of 1.80. Different amounts of AlCl₃ (32° Be) solution were added to the above 33% BAC solution, and the resultant solutions were spray dried. Experiment Number 7 was dried the next day and Experiment Number 8, after two days.

TABLE III

| Experiment | | % | | | | |
|---|---|---|---|---|---|---|
| Number | Al/Cl Ratio | A.S. | % Band I | III/II | % IV | % III + IV |
| 7 | 1.62 | 81.3 | 4.4 | 0.97 | 39.5 | 67.1 |
| 8 | 1.24 | 76.4 | 0 | 0.94 | 66.1 | 82.5 |

### EXAMPLE 4

### Preparation of Double Activated Aluminum Salt Compositions by Utilizing the Method Referenced as Process C

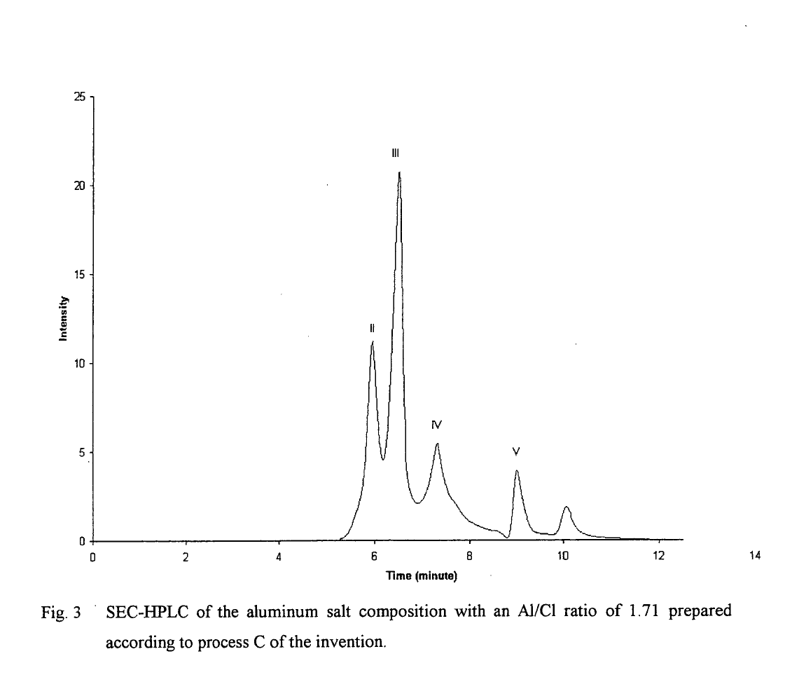

Four double activated aluminum salt solutions were made through the reaction of Al powder and AlCl₃ aqueous solution as described in Experiment 3, and part of the solutions were spray dried the next day by using a two fluid nozzle at an outlet temperature of about 210° F. Some losses on chloride have been observed especially at lower Al/Cl ratios. The results are summarized in Table IV, which characterizes the solutions, and Table V, which characterizes the powders (dried solution “d”) and shows some loss of the chloride during the spray drying process. The SEC-HPLC of the Al salt composition according to Experiment Number 9d is shown in FIG. 3.

TABLE IV

| Experiment | | | | | | |
|---|---|---|---|---|---|---|
| Number | Al/Cl Ratio | % Al | % Band I | III/II | % IV | % III + IV |
| 9 | 1.67 | 7.46 | 0 | 1.89 | 24.4 | 73.8 |
| 10 | 1.54 | 7.18 | 0 | 2.14 | 39.8 | 80.8 |
| 11 | 1.49 | 7.67 | 0 | 0.87 | 41.7 | 68.9 |
| 12 | 1.35 | 7.17 | 0 | 1.10 | 49.7 | 76.1 |

TABLE V

| Experiment | | % | | | | |
|---|---|---|---|---|---|---|
| Number | Al/Cl Ratio | A.S. | % Band I | III/II | % IV | % III + IV |
| 9d | 1.71 | 81.56 | 0 | 1.64 | 25.9 | 71.9 |
| 10d | 1.63 | 81.39 | 0 | 1.66 | 33.5 | 75.0 |
| 11d | 1.55 | 81.91 | 0 | 0.94 | 39.0 | 68.6 |
| 12d | 1.47 | 80.20 | 0 | 0.95 | 46.9 | 72.8 |

Each of the novel Al salt compositions according to the present invention have pH (15% w/w) values above 3.5 as illustrated in Table VI, and as such, afford skin friendly antiperspirant vehicles when formulated in aerosol, stick and soft solid products.

TABLE VI

| | Experiment Number | | | | | | | | | |
|---|---|---|---|---|---|---|---|---|---|---|
| | 1 | 2 | 3 | 4 | 7 | 8 | 9d | 10d | 11d | 12d |
| pH (15% w/w) | 4.3 | 3.9 | 3.8 | 3.7 | 4.0 | 3.7 | 4.0 | 3.9 | 3.8 | 3.8 |

It will be understood that the present invention is susceptible to numerous changes and modifications as apparent to those skilled in the art. Accordingly, the present invention may be embodied in other specific forms without departing from the spirit of essential attributes of the invention disclosed herein and reference should be made to the appended claims, in the light of the foregoing specifications, as indicating the scope of the invention.

What is claimed is:

1. An aluminum antiperspirant composition of enhanced efficacy
(a) having the formula
Al₂(OH)₆₋ₐXₐ
wherein X is Cl⁻, Br⁻, l⁻, or NO₃⁻, a is from about 1.1 to about 1.7, and
(b) characterized by exhibiting an SEC-HPLC Band I area of less than 10%, a Band III of at least 20%, a Band III/II ratio of at least 0.7, a Band IV area of at least 20%.

2. The composition of claim 1 wherein X is chloride.

3. The antiperspirant composition of claim 1 wherein the aluminum to halide atomic ratio is from about 1.4 to about 1.6.

4. The composition of claim 1 wherein the SEC-HPLC Band I area is less than about 5%, the Band III area is at least 30%, and the Band IV area is at least 25%, and at least 60% of the aluminum species is contained in Bands III and IV.

5. A dried powder antiperspirant having the composition of claim 1.

6. A method of preparing an antiperspirant having the composition of claim 1 by a means selected from process steps:
(a) heating and refluxing a BAC solution having an aluminum/halide ratio above about 1.5 and a concentration of from about 10% to about 25% by weight until an HPLC Band III/II ratio of at least 0.7 is obtained, cooling the reaction product and adding thereto a strong acid;
(b) making a BAC solution by adding aluminum powder to an aqueous AIX₃ solution wherein X is Cl⁻, Br⁻ and I⁻ at reaction temperature of about 80° C. to obtain a reaction product having an aluminum/halide ratio of from about 1.75 to about 1.95 and a concentration of about 25% to 35% by weight and filtering the reaction product to obtain a clear solution and reacting said filtered solution with a strong acid; and
(c) preparing a BAC solution by reacting aluminum powder directly with an aqueous AIX₃ solution wherein X is Cl⁻, Br⁻ and I⁻ at reaction temperature of about 80° C. to obtain a reaction product having an aluminum/halide ratio of from about 1.2 to about 1.8 and a concentration of about 30% to 35% by weight.

7. A method of preparing an antiperspirant according to process step (a) of claim 6.

8. A method of preparing an antiperspirant according to process step (b) of claim 6.

9. A method of preparing an antiperspirant according to process step (c) of claim 6.

10. The method of claim 6 wherein the strong acid is AlCl₃.

11. The method of claim 6 wherein the strong acid is hydrochloric acid.

12. The method of claim 6 wherein the aluminum/halide atomic ratio of the composition is from about 1.4 to about 1.7.

13. The method of claim 7 wherein the BAC solution is 20% by weight.

14. The method according to claim 6 wherein the reaction product of the process steps (a), (b) and (c) is subjected to a drying operation.

15. The method according to claim 9 wherein the concentration of the solution is about 30% by weight.

16. The method of claim 8 wherein AlX₃ is AlCl₃.

17. The method of claim 9 wherein the aluminum to chloride atomic ratio of the composition is from about 1.4 to about 1.7.

18. The method according to claim 8 wherein the concentration of the solution is about 30% by weight.

19. An antiperspirant product selected from creams, aerosols, soft solids and solid sticks comprising the powder composition of claim 5.
